(19) United States
(12) Patent Application Publication
(10) Pub. No.: US 2006/0134638 A1
(43) Pub. Date: Jun. 22, 2006

(54) Error reduction in automated gene synthesis

(75) Inventors: John T. Mulligan, Seattle, WA (US); John C. Tabone, Bothell, WA (US)

Correspondence Address: SEED INTELLECTUAL PROPERTY LAW GROUP PLLC, 701 FIFTH AVE, SUITE 6300, SEATTLE, WA, 98104-7092, US

(73) Assignee: Blue Heron Biotechnology, Inc., Bothell, WA (US)

(21) Appl. No.: 10/816,459

(22) Filed: Apr. 1, 2004

Related U.S. Application Data
(60) Provisional application No. 60/460,021, filed on Apr. 2, 2003.
(60) Provisional application No. 60/488,455, filed on Jul. 18, 2003.

(51) Int. Cl. C12Q 1/68; C12P 19/34

(52) U.S. Cl. 435/6; 435/91.2

(57) ABSTRACT

In embodiments of the present invention, methods are provided for removing double-stranded oligonucleotide (e.g., DNA) molecules containing one or more sequence errors, generated during nucleic acid synthesis, from a population of correct oligonucleotide duplexes. In one embodiment, the oligonucleotides are generated enzymatically. Heteroduplex (containing mismatched bases) oligonucleotides may be created by denaturing and reannealing the population of duplexes. The reannealed oligonucleotide duplexes are contacted with a mismatch recognition protein that interacts with (e.g., binds and/or cleaves) the duplexes containing a base pair mismatch. The oligonucleotide heteroduplexes that have interacted with such a protein are separated, simultaneously with contacting or sequentially in a separate step, from homoduplexes. These methods are also used in another embodiment to remove heteroduplex oligonucleotides (e.g., DNA) that are formed directly from chemical nucleic acid synthesis. In other embodiments of the present invention, kits and compositions useful for the methods are provided.

### CROSS-REFERENCE TO RELATED APPLICATIONS

This application claims the benefit of U.S. Provisional Patent Application Nos. 60/460,021, filed Apr. 2, 2003, and 60/488,455, filed Jul. 18, 2003, which applications are incorporated herein in their entirety.

### BACKGROUND OF THE INVENTION

1. Field of the Invention

The present invention in certain embodiments is directed toward the removal of double-stranded oligonucleotides containing sequence errors. It is more particularly related to the removal of error-containing oligonucleotides (such as error-containing double-stranded DNA), generated for example by chemical or enzymatic synthesis (including by PCR amplification), by removal of mismatched duplexes using mismatch recognition proteins. The invention in other embodiments relates to kits and compositions useful for the methods of the invention.

2. Description of the Related Art

For purposes of this application, DNA is used as a prototypical example of an oligonucleotide. Mismatches are formed directly during chemical DNA synthesis or are formed in enzymatically synthesized DNA by denaturing and reannealing a mixed population of correct and error-containing DNA.

In chemical DNA synthesis, the mismatches originate during the synthesis of oligonucleotides (“oligos”). These oligos are used as building blocks for DNA synthesis and are synthesized as single strands using automated oligonucleotide synthesizers. Random chemical side reactions create base errors in these single-stranded oligos. When two complementary synthetic oligos are hybridized to form double-stranded DNA, there is almost no chance that the random base errors formed in one strand will be correctly base paired in the opposite strand. It is these incorrectly paired bases that form the mismatches found in chemically synthesized double-stranded DNA.

In enzymatic DNA synthesis, an enzyme (such as a polymerase) is used to amplify or assemble from a synthetic DNA template. This template contains the same type of base mismatches that are found in the synthetic DNA described above. However, once this DNA is amplified, the mismatches are converted into base paired errors in sequence. These base pairings of the mismatches occur as polymerase synthesizes the complementary base on the strand opposing strand. The result of this enzymatic step is to create a mixed population of DNA molecules where all bases are paired correctly with both correct (error-free) and incorrect (error-containing) sequences. The polymerase step essentially maintains the ratio of correct to incorrect sequence.

A DNA population such as that formed from enzymatic DNA synthesis containing both error-free and error-containing base paired DNA where both are correctly base pair matched, can be converted to a population composed of both mismatched and error-free correctly base paired DNA by denaturation and reannealing. When these steps are performed on a population that contains a small fraction of error-containing molecules relative to correct molecules, the vast majority of error containing strands will hybridize with the more abundant correct strand and will form mismatched sites.

Moreover, even if the errors represent a high fraction of the population (e.g., 50%) denaturation and reannealing of a DNA population to itself, will result in the vast majority of a particular error-containing strand hybridizing either to a correct strand or to a strand that contains a distinct error. Thus, a population of DNA will be converted into two populations of mostly base paired correct DNA. The correct strands will find correct strand complementary strands and form perfectly base paired duplexes.

Gene synthesis is a method of producing gene-sized DNA clones by assembling chemically synthesized oligonucleotides into larger fragments and then cloning these fragments. Gene synthesis improves the productivity of biological research by allowing scientists to spend more time on experiments and less time on “cutting and pasting” genes. The ability to design and acquire any DNA molecule also facilitates new approaches to understanding gene function and allows researchers to build genes with entirely novel functions.

One critical limitation on gene synthesis technology is the error rate. Cloned, chemically-synthesized DNA fragments have a sequence error every 200 to 500 bp on average. The most common mutations in oligonucleotides are deletions that can come from capping, oxidation and/or deblocking failure. Other prominent side reactions include modification of guanosine (G) by ammonia to give 2,6-diaminopurine, which codes as an adenosine (A). Deamination is also possible with cytidine (C) forming uridine (U) and adenosine forming inosine (I).

Each strand is produced separately, and thus the errors are statistically independent. This approach results in most errors being paired with the correct sequence, leading to the formation of a heteroduplex molecule. For large genes, current error rates make direct cloning of the gene impractical. In effect, the error rate puts an upper limit on the size of an accurate fragment that can be cloned in an economical way.

The error rate also limits the value of gene synthesis for the production of libraries of gene variants. With an error rate of 1/300, only 0.7% of the clones in a 1500 base pair gene will be correct. As most of the errors from oligonucleotide synthesis result in frame-shift mutations, over 99% of the clones in such a library will not produce a full-length protein. Reducing the error rate by 75% would increase the fraction of clones that are correct by a factor of 40.

Due to the difficulties in the current approaches to the preparation or amplification of oligonucleotides, such as genes, there is a need in the art for methods for improving the removal of double-stranded oligonucleotides containing sequence errors. For example, there is a need in the art to reduce the error frequency of synthetic DNA fragments in order to facilitate the synthetic production of large DNA fragments including genes and gene variant libraries. The present invention fills this need by improving upon current gene synthesis error frequencies, and further provides other related advantages.

### BRIEF SUMMARY OF THE INVENTION

Briefly stated, in certain embodiments the present invention provides a variety of methods, compositions and kits for removing double-stranded oligonucleotide (e.g., DNA) molecules containing one or more sequence errors generated during nucleic acid synthesis, from a population of correct oligonucleotide duplexes. In one embodiment, the oligonucleotides are generated enzymatically. Heteroduplex oligonucleotides may be created by denaturing and reannealing the population of duplexes. The reannealed oligonucleotide duplexes are contacted with a mismatch recognition protein that interacts with the duplexes containing a base pair mismatch. The oligonucleotide heteroduplexes that have interacted with the protein are separated from homoduplexes as the latter do not interact with the protein. These methods are also used to remove heteroduplex oligonucleotides (e.g., DNA) that are formed directly from chemical nucleic acid synthesis.

In one embodiment, the present invention provides a method of depleting in a sample of double-stranded oligonucleotides a population of double-stranded oligonucleotides containing mismatched bases thereby enriching in said sample a population of double-stranded oligonucleotides containing correctly matched bases, comprising the steps of: (a) contacting said sample containing double-stranded oligonucleotides with a mismatch recognition protein under conditions to permit the protein to interact with a double-stranded oligonucleotide containing at least one mismatched base; and (b) collecting double-stranded oligonucleotides that have not interacted with said mismatch recognition protein, thereby depleting the population of double-stranded oligonucleotides containing mismatched bases. In another embodiment, there is, prior to the step of collecting, an additional step comprising separating said double-stranded oligonucleotide containing at least one mismatched base that has interacted with said mismatch recognition protein, from double-stranded oligonucleotides that have not interacted with said mismatch recognition protein. In another embodiment, there is, immediately following step (a) or simultaneous with step (a), an additional step comprising contacting the sample with a nucleotide containing biotin under conditions to permit incorporation of the nucleotide into the oligonucleotides that have interacted with the mismatch recognition protein. In another embodiment, there is, following the step of contacting the sample with a nucleotide containing biotin, an additional step comprising contacting said sample with an avidin under conditions to permit the avidin to interact with the biotin. The avidin may be immobilized on a solid support.

In another embodiment, the present invention provides a kit for depleting double-stranded oligonucleotides containing mismatched bases from a population of double-stranded oligonucleotides, comprising a mismatch recognition protein, buffer, control oligonucleotides and instructions. The kit may further comprise material for separating mismatch protein bound oligonucleotides from unbound oligonucleotides. In preferred embodiments, the double-stranded oligonucleotides are double-stranded DNA. In particularly preferred embodiments, the double-stranded DNA is a gene or a portion of a gene.

These and other aspects of the present invention will become evident upon reference to the drawings and the following detailed description. In addition, various references are set forth herein. Each of these references is incorporated herein by reference in its entirety as if each was individually noted for incorporation.

### BRIEF DESCRIPTION OF THE DRAWINGS

FIG. 1 shows the results of a Taq MutS gel shift assay.

FIG. 2A depicts heteroduplex DNA containing a single A or T bulges were created by denaturing and reannealing 410 bp fragments of pUC119 and pUC120. Cleavage with Sapl and Sfol cleaved homoduplex molecules and allowed recovery of heteroduplex LacZ+ fragments. (SEQ ID NOS. 1-6).

FIG. 2B depicts the generation of pUC121 with a stop codon in frame it the 5′ coding region such that the 410 bp AflIII/EcoRI fragment is lacZ- when ligated into pUC119. (SEQ ID NOS. 1-2).

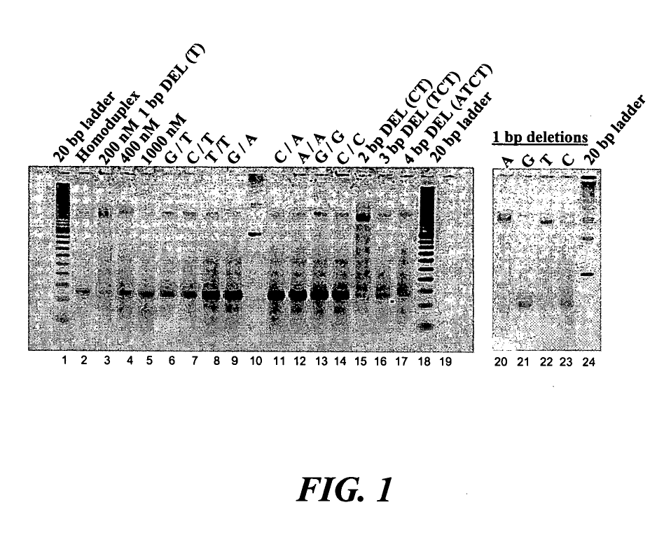

### DETAILED DESCRIPTION OF THE INVENTION

Prior to setting forth the invention, it may be helpful to an understanding thereof to set forth definitions of certain terms to be used hereinafter.

Natural bases of DNA—adenine (A), guanine (G), cytosine (C) and thymine (T). In RNA, thymine is replaced by uracil (U).

Synthetic double-stranded oligonucleotides—two strands of oligonucleotides (e.g., substantially double-stranded DNA) composed of single strands of oligonucleotides synthetically produced (e.g., by chemical synthesis or by the ligation of synthetic double-stranded oligonucleotides to other synthetic double-stranded oligonucleotides to form larger synthetic double-stranded oligonucleotides) and joined together in the form of a duplex.

Synthetic failures—undesired products of oligonucleotide synthesis; such as side products, truncated products or products from incorrect ligation.

Side products—chemical byproducts of oligonucleotide synthesis.

Truncated products—all possible shorter than the desired length oligonucleotide, e.g., resulting from inefficient monomer coupling during synthesis of oligonucleotides.

TE—an aqueous solution of 10 mM Tris and 1 mM EDTA, at a pH of 8.0.

Homoduplex oligonucleotides—double-stranded oligonucleotides wherein the bases are fully matched; e.g., for DNA, each A is paired with a T, and each C is paired with a G.

Heteroduplex oligonucleotides—double-stranded oligonucleotides wherein the bases are mispaired, i.e., there are one or more mismatched bases; e.g., for DNA, an A is paired with a C, G or A, or a C is paired with a C, T or A, etc.

Mismatch recognition protein—a protein that recognizes heteroduplex oligonucleotides (e.g., heteroduplex DNA); typically the protein is a mismatch repair enzyme or other oligonucleotide binding protein (e.g., DNA mismatch repair enzyme or other DNA binding protein); the protein may be isolated or prepared synthetically (e.g., chemically or enzymatically), and may be a derivative, variant or analog, including a functionally equivalent molecule which is partially or completely devoid of amino acids.

The present invention is directed in certain embodiments toward methods, compositions and kits for the removal of error-containing double-stranded oligonucleotide (e.g., DNA) molecules from a population of double-stranded oligonucleotides (e.g., that are produced by chemical or enzymatic synthesis). The error-containing oligonucleotide molecules in this population are removed from the correct molecules when the errors are present as mismatches in the double-stranded oligonucleotides. The removal of the mismatch is based in the present invention on the use of mismatch recognition proteins that recognize mismatched bases in double-stranded oligonucleotides. Such proteins interact with double-stranded oligonucleotides containing mismatched bases (e.g., by binding and/or cleaving on or near the mismatch site). The protein step may or may not be performed in conjunction with a separation step (e.g., chromatographic step) to separate mismatch-containing heteroduplex from homoduplex oligonucleotides. It is to be understood that the methods of the invention have the capability of mismatch removal regardless of the way the mismatch was created in the population.

More specifically, the disclosure of the present invention shows surprisingly that mismatch recognition proteins may be used to deplete an oligonucleotide population of those double-stranded oligonucleotides which contain sequence errors. Depletion of error-containing oligonucleotides from the desired double-stranded oligonucleotides refers generally to at least about (wherein “about” is within 10%) a two-fold depletion relative to the total population prior to separation. Typically, the depletion will be a change of about two-fold to three-fold from the original state. The particular fold depletion may be the result of a single use of the method (e.g., single separation) or the cumulative result of a plurality of use (e.g., two or more separations). Depletion of error-containing oligonucleotides is useful, for example, where the oligonucleotides are double-stranded DNA which correspond to a gene or fragments of a gene.

Oligonucleotides suitable for use in the present invention are any double-stranded sequence. Examples of such oligonucleotides include double-stranded DNA, double-stranded RNA, DNA/RNA hybrids, and functional equivalents containing one or more non-natural bases. Preferred oligonucleotides are double-stranded DNA. Double-stranded DNA includes full length genes and fragments of full length genes. For example, the DNA fragments may be portions of a gene that when joined form a larger portion of the gene or the entire gene.

The present invention in certain embodiments provides methods that selectively remove double-stranded oligonucleotides, such as DNA molecules, with mismatches, bulges and small loops, chemically altered bases and other heteroduplexes arising during the process of chemical synthesis of DNA, from solutions containing perfectly matched synthetic DNA fragments. The methods separate specific protein-DNA complexes formed directly on heteroduplex DNA or through avidin-biotin-DNA complexes formed following the introduction of a biotin molecule into heteroduplex containing DNA and subsequent binding by any member of the avidin family of proteins, including streptavidin. The avidin may be immobilized on a solid support. A minimum of one incorporated biotin is sufficient to label the strand for avidin binding. Typically all the normal nucleotides are included in addition to the biotin labeled nucleotide in order to facilitate labeling of all possible nick positions.

Central to the method are enzymes that recognize and bind specifically to mismatched, or unpaired bases within a double-stranded oligonucleotide (e.g., DNA) molecule and remain associated at or near to the site of the heteroduplex, create a single or double strand break or are able to initiate a strand transfer transposition event at or near to the heteroduplex site. The removal of mismatched, mispaired and chemically altered heteroduplex DNA molecules from a synthetic solution of DNA molecules results in a reduced concentration of DNA molecules that differ from the expected synthesized DNA sequence and thus a greater yield of correct clones when introduced into a plasmid and transformed into a cell.

As noted above, the present invention provides a preparative method to remove base mismatched oligonucleotides from a population of correctly base matched oligonucleotides. The method generally comprises the steps of contacting a double-stranded oligonucleotide sample with a mismatch recognition protein, and collecting the double-stranded oligonucleotides that have not interacted with the mismatch recognition protein. Collecting the double-stranded oligonucleotides that have not interacted with the protein can be the result of their removal from the sample, or the removal from the sample of those oligonucleotides that did interact. The step of contacting is performed under conditions (including a time sufficient) to permit a mismatch recognition protein to interact with (e.g., bind to and/or cleave) mismatch-containing heteroduplex oligonucleotides. The method may, prior to the step of collecting, optionally include a step of separating the double-stranded oligonucleotide that contains at least one (one or more) mismatched base and that has interacted with the mismatch recognition protein, from double-stranded oligonucleotides that have not interacted with the mismatch recognition protein. The method may, in place of or in addition to a separation step and prior to the step of contacting, optionally include steps of first denaturing and then reannealing a sample of double-stranded oligonucleotides under conditions to permit conversion of the double-stranded oligonucleotides first to single-stranded oligonucleotides and then to double-stranded oligonucleotides. It will be evident to one of ordinary skill in the art that the steps may be performed sequentially, or two or more steps may be performed simultaneously. For example, in an embodiment where a mismatch recognition protein is immobilized on a solid support, the step of contacting results directly in separation.

In one embodiment the mismatch recognition proteins share the property of binding on or within the vicinity of a mismatch. Such a protein reagent includes proteins that are endonucleases, restriction enzymes, ribonucleases, mismatch repair enzymes, resolvases, helicases, ligases and antibodies specific for mismatches. Variants of these proteins can be produced, for example, by site directed mutagenesis, provided that they are functionally equivalent for mismatch recognition. The enzyme can be selected, for example, from T4 endonuclease 7, T7 endonuclease 1, S1, mung bean endonuclease, MutY, MutS, MutH, MutL, cleavase, and HINF1. In another embodiment of the invention, the mismatch recognition protein cleaves at least one strand of the mismatched DNA in the vicinity of the mismatch site.

In the case of proteins that tightly bind specifically and directly to heteroduplex sites, for example members of the ubiquitous MutS family of proteins, the protein-DNA complex formed is separated from the unbound perfectly matched DNA duplexes. The MutS enzyme family functions to bind mismatches and unpaired bases in vivo, as an early step in repairing replication misincorporation or slippage errors arising during DNA synthesis in vivo. The property of specific heteroduplex recognition is exploited in the present invention to remove DNA molecules containing heteroduplex sites from a synthetic pool of perfectly matched and heteroduplex containing DNA molecules

In the case of proteins that recognize and cleave heteroduplex DNA forming a single strand nick, for example the CELI endonuclease enzyme, the resultant nick can be used as substrate for DNA polymerase to incorporate modified nucleotides containing a biotin moiety. There are many examples of proteins that recognize mismatched DNA and produce a single strand nick, including resolvase endonucleases, glycosylases and specialized MutS-like proteins that posses endonuclease activity. In some cases the nick is created in a heteroduplex DNA molecule after further processing, for example the thymine DNA glycosylases recognize mismatched DNA and hydrolyze the bond between deoxyribose and one of the bases in DNA, generating an abasic site without necessarily cleaving the sugar phosphate backbone of DNA. The abasic site can be converted by an AP endonuclease to a nicked substrate suitable for DNA polymerase extension. Protein-heteroduplex DNA complexes can thus be formed directly, in the example of MutS proteins, or indirectly following incorporation of biotin into the heteroduplex containing strand and subsequent binding of biotin with streptavidin or avidin proteins.

In another embodiment of the invention, transposase enzymes such as the MuA transposase preferentially inserts biotin labeled DNA containing a precleaved version of the transposase DNA binding site into or near to the site of mismatched DNA in vitro via a strand transfer reaction. The in vitro MuA transposase directed strand transfer is known by those skilled in the art and familiar with transposase activity to be specific for mismatched DNA. The precleaved MuA binding site DNA is known by those who work with MuA transposase to consist of a minimal region of 51 bp of DNA. In this method, the precleaved MuA binding site DNA is biotinylated at the 5′ end of the molecule enabling the formation of a protein-biotin-DNA complex with streptavidin or avidin protein following strand transfer into heteroduplex containing DNA.

The optional separation step can be performed in a variety of means, e.g., using high performance liquid chromatography (HPLC), by size exclusion chromatography, ion exchange chromatography, affinity chromatography or reverse phase chromatography. The separation can also be performed using membranes in a slot blot fashion or a microtiter filter plate. The separation may also be performed using solid phase extraction cartridges using supports similar to the HPLC columns.

Separation of protein-DNA complexes in vitro can be achieved by incubation of the solution containing protein-DNA complexes with a solid matrix that possesses high affinity and capacity for binding of protein and low affinity for binding of DNA. One example of such a solid protein binding matrix is the commercially available protein binding spin filtration columns marketed as alternatives for phenol chloroform extractions for removal of molecular biology protein reagents such as restriction endonucleases from DNA solutions. Another example of a solid protein binding matrix is a hydroxylated resin marketed by Stratagene as a product to remove unwanted proteins from DNA preparations. Protein-DNA complexes can be separated from DNA molecules that are not associated with protein by exposing the synthetic DNA solution containing the protein-DNA complexes to synthetic membranes or solid resin supports that possess high affinity and capacity for binding of proteins or for the specific protein and low affinity for binding of DNA and filtering or centrifuging the solution through these membranes and collecting the deproteinized eluate enriched for perfectly matched DNA molecules.

In embodiments, a mismatch recognition protein (e.g., the MutS protein from E. coli) or an avidin is immobilized on a solid support. Methods for immobilizing molecules on solid supports are well known to one in the art, and include covalent or noncovalent attachment to a solid support. Similarly, types of suitable solid supports are well known to one in the art, and include beads, glass, polymers, resins and gels. The following is a representative example for preparing oligonucleotides depleted of error-containing oligonucleotides. Two complementary oligonucleotides (e.g., DNA) are chemically synthesized and then hybridized to form duplex oligonucleotides (e.g., double-stranded DNA). Alternatively, double-stranded DNA may be enzymatically synthesized (and further denatured and reannealed). This mixture is passed over a column with a mismatch recognition protein (e.g., the MutS protein) immobilized on a solid support (such as beads) in the column. Fragments with an error in either of the oligonucleotides will usually contain a mismatch since in most cases the other strand is correct at that position. Duplexes containing mismatches will bind to the column and only error-free duplexes will be enriched in the flow-through from the column.

In another embodiment, a gene encoding a mismatch recognition protein (e.g., the MutS gene) is fused to a gene fragment that encodes a binding domain (for instance a chitin-binding domain). The following is another representative example for preparing oligonucleotides depleted of error-containing oligonucleotides. The fused protein is produced and mixed with a duplex fragment that is produced as described above. Duplex molecules with an error in either strand will bind to the fusion protein (e.g., MutS fusion protein). After an appropriate incubation, the mixture is passed over a chitin column. The fusion protein binds to the column via the chitin. Duplex molecules with mismatches are retained on the column, and error-free duplexes flow through.

In another embodiment, the present invention provides kits for removing mismatch-containing molecules from a population of synthetic molecules. The kits contain one or more of the mismatch recognition proteins required for carrying out the subject methods. Kits may contain reagents in pre-measured amounts so as to ensure both precision and accuracy when performing the subject methods. Kits may also contain instructions for performing the methods of the invention. Typically, the kits contain a mismatch recognition protein, buffer, control oligonucleotides (e.g., DNA) and instructions. The kit optionally further comprises material for separating the mismatch protein bound oligonucleotides (e.g., DNA) from unbound oligonucleotides (e.g., DNA). In a preferred embodiment, the kits contain MutS protein and a material that binds MutS protein but not unbound oligonucleotides (e.g., DNA). In some preferred embodiments, the kits may contain biotin nucleotides, a polymerase (e.g., DNA polymerase) and a biotin binding protein. In other preferred embodiments, the kits contain MuA transposase, a Mu end DNA fragment, and a method for separating the Mu end DNA fragment from a mixture of other DNA fragments.

Experimental Tools

a. Synthetic Duplexes. A set of 50 bp duplexes is used as a first test of activity for most of the schemes described below. The set includes a homoduplex, all eight native base heteroduplexes (A/A, A/C, A/G, T/T, T/C, T/G, C/C, and G/G), three unnatural base heteroduplexes (diamino purine/C, deoxyuridine/G, and Inosine/T), and all four one base pair deletions (-/A, -/T, -/C, and -/G).

b. Synthetic Fragment. A large batch of oligonucleotides is prepared for synthesis of a standard 400 bp fragment. The same materials are used to test each of the error-reduction techniques. To simplify the error analysis, the fragment has been designed to yield high quality sequence. Two versions are prepared, one with fully complimentary oligonucleotides and one with an A deletion in the center, yielding a T bulge.

c. Nicked Fragment. The test fragment sequence includes an N.BbvC IA site; digestion with this nicking endonuclease produces a single nick near the center of the plus strand. This provides a model for the mismatch-dependent nicks produced by a number of the test treatments.

d. Sequencing. Cloning and sequencing the synthetic 400 bp fragment are the primary experimental output used to judge the effectiveness of the error-removal methods. For each condition, 96 clones are sequenced, yielding an average of approximately 150 errors per test.

e. Commercially Available DNA Repair Enzymes.

TABLE 1
A partial list of commercially available repair enzymes
and their sources. Commercial sources: NEB, New England
Biolabs; R&D, R&D Systems; USB, US Biologicals.

| Enzyme | Activity | Source |
|---|---|---|
| E. coli Endonuclease III | DNA | Trevigen, NEB |
| | glycosylase | |
| | and AP lyase | |
| E. coli Endonuclease IV | AP endonuclease, | Trevigen |
| E. coli Uracil-N- | DNA | Trevigen, NEB |
| Glycosylase | glycosylase | |
| Murine 3-Methyladenine | DNA | Trevigen |
| Glycosylase | glycosylase | |
| E. coli MutY Enzyme | DNA | Trevigen, R&D |
| | glycosylase | |
| | and AP lyase | |
| Thermostable TDG | DNA | Trevigen, R&D |
| | glycosylase | |
| E. coli Endonuclease VIII | DNA | Trevigen |
| | glycosylase, | |
| | AP | |
| | endonuclease | |
| Human 8-oxo-Guanine | DNA glycosylase | Trevigen, NEB |
| DNA Glycosylase | | |
| Fpg Protein | Formamido- | Trevigen, NEB |
| | pyrimidine- | |
| | DNA | |
| | glycosylase | |
| | and AP lyase | |
| Exonuclease III | AP endonuclease | NEB, others |
| T7 Endonuclease I | Cleaves within | NEB |
| | 6 bp of | |
| | mismatches | |
| E. coli Endonuclease V | Cleaves 3′ | Trevigen, R&D |
| | to mismatches | |
| T4 Endonuclease VII | Cleaves near | Amersham |
| | mismatches | |
| Cell | Cleaves at | Transgenomics* |
| | mismatches | |
| UVDE | Cleaves 5′ of | R&D |
| | a variety of | |
| | photoadducts | |
| E. coli MutS | Mismatch | USB, |
| | binding, cleavage | Genecheck |
| E. coli MutL | Mismatch | USB, |
| | cleavage complex | Genecheck |
| E. coli MutH | Mismatch | USB, |
| | cleavage complex | Genecheck |
| Taq MutS | Mismatch binding | Epicentre** |

*Cell is available as a sample from Transgenomics.
**Taq MutS is available on a limited basis from Epicentre, but is not in their catalogue or on their web site.

System for Economical Quantitative Measurement of Low Error Rates:

Vector and synthetic fragment for error detection. The lac repressor (lacl) cloning strategy shown below is to allow the quantitative measurement of low error rates. The cloned synthetic fragment carries two functions: 1) a promoter and 300 bp of the lacl gene, and 2) a promoter for the chloramphenicol resistance gene. The lad gene is well characterized and simple to detect using a colorimetric assay. The first 60 amino acid residues of the protein comprise a DNA binding domain; most or all changes in 28 of the amino acid residues in this domain lead to an inactive repressor.

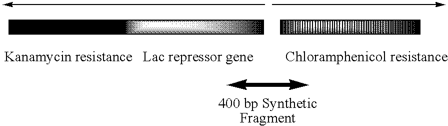

Using this system, each transformed colony permits detection of deletions in 300 base pairs of synthetic DNA and substitutions in approximately 60 base pairs of synthetic DNA. Selection for chloramphenicol resistance ensures that each clone carries the synthetic DNA fragment. In a bacterial strain with beta-galactosidase under the control of the lac repressor, clones with the correct sequence form white, kanamycin-resistant colonies. Clones with a substitution in one of the 60 critical bases in the lad gene form blue, kanamycin-resistant colonies. Clones with a deletion in the 300 bp of the lac repressor open reading frame form blue, kanamycin-sensitive colonies. Each colony represents 60 bp of high-quality sequence information. By counting blue colonies on chloramphenicol plates, the rate of deletions can be measured. By counting blue colonies on kanamycin plates, the rate of substitutions can be measured.

Physical Separation of Heteroduplex and Homoduplex DNA.

Three techniques are tested for physically separating heteroduplex molecules from the population of synthetic DNA; 1) DHPLC, 2) binding by MutS protein and 3) binding by TDG.

DHPLC. Partially denaturing high performance liquid chromatography (DHPLC) has established itself as a powerful tool for DNA variation screening and allele discrimination (1). At temperatures where DNA molecules are fully duplexed, reverse-phase HPLC using commercially-available columns can separate DNA fragments by length with high resolution. At elevated temperatures, heteroduplexes will partially denature and show reduced retention times relative to fully-duplexed molecules. At the appropriate temperature they appear as distinct peaks from homoduplex molecules of the same size. The temperature at which heteroduplex and homoduplex molecules can be distinguished depends on the sequence and length of the molecule and can be predicted using software available from Stanford University (http://insertion.stanford.edu/melt.html). DHPLC is used in combination with the digestion methods described below.

MutS binding. The MutSLH proteins are the central elements of mismatch repair in E. coli. The MutS gene product binds to mismatches and, in combination with MutL, signals MutH to nick the DNA on the newly-synthesized strand. This initiates the removal of a large section of DNA of the newly-synthesized strand in a reaction that involves Helicase II and an exonuclease, and the subsequent re-synthesis of that region to repair the mismatch. Members of the MutS family are a fundamental part of the cell's ability to preserve genetic fidelity, and are present in most or all free-living organisms. MutS binding has been described as a method for in vitro detection of SNPs by altering the mobility of heteroduplex DNA in gel-shift experiments (2). MutS gel-shift assays are used herein to separate heteroduplex molecules from homoduplex molecules. Both E. coli MutS and Taq MutS are tested, as the latter is reported to show greater discrimination between heteroduplex and homoduplex DNA (3).

TDG binding. Pan and Weissman (4) described the use of thymine DNA glycosylases (TDGs) to enrich mismatch-containing or perfectly-matched DNA populations from complex mixtures. DNA glycosylases hydrolyze the bond between deoxyribose and one of the bases in DNA, generating an abasic site without necessarily cleaving the sugar phosphate backbone of DNA. Pan and Weissman found that all four groups of single base mismatches and some other mismatches could be hydrolyzed by a mixture of two TDGs. In addition, their data showed that in the absence of magnesium the enzymes exhibit a high affinity for abasic sites, and could thus be used to separate DNA molecules into populations enriched or depleted for heteroduplexes.

Preferential Digestion of Heteroduplex Molecules.

Several large classes of enzymes preferentially digest DNA substrates containing mismatches, deletions or damaged bases. Each of these enzymes acts to convert their damaged or mismatched substrates into nicks or single base pair gaps (in some cases with the help of an AP endonuclease that converts abasic sites into nicks). Three classes of enzymes are tested for their utility in modifying synthetic fragments which contain errors: DNA glycosylases, mismatch endonucleases, and the MutSLH mismatch repair proteins. Each is described in more detail below.

The present invention uses these nicks or small gaps to identify the error-containing DNA molecules and remove them from the cloning process. A combination of techniques are tested for removing the nicked DNA, including Exonuclease III (Exo III) digestion, HPLC separation, and direct cloning.

DNA Glycosylases. DNA glycosylases are a class of enzymes that remove mismatched bases and, in some cases, cleave at the resulting apurinic/apyrimidimic (AP) site. A very large number of DNA glycosylases have been identified, and at least eight are commercially available (see Table 1). They typically act on a subset of unnatural, damaged or mismatched bases, removing the base and leaving a substrate for subsequent repair. As a class, the DNA glycosylases have broad, distinct and overlapping specificities for the chemical substrates that they will remove from DNA. Although glycosylase treatment may not remove the most common errors in synthetic DNA (short deletions), these enzymes may be useful in reducing the error rate to low levels. Well-known side reactions in oligonucleotide synthesis chemistry such as capping failure and deamination can account for many of the common sequence errors, but lower-frequency errors may result from unknown mechanisms. A large set of glycosylases are tested for the present invention because their range of specificities gives them the potential to remove as yet unknown sources of sequence errors in synthetic DNA. Glycosylases that leave AP sites are combined with an AP endonuclease such as E. coli Endonuclease IV or Exo III to generate a nick in the DNA.

Mismatch endonucleases. Seven commercially available enzymes are reported to nick DNA in the region of mismatches or damaged DNA: T7 Endonuclease I, E. coli Endonuclease V, T4 Endonuclease VII, mung bean nuclease, Cell, E. coli Endonuclease IV and UVDE. Endo IV is identified as an AP endonuclease in the supplier's description, but was recently reported to nick DNA on the 5′ side of various oxidatively-damaged bases (5).

MutSLH Digestion. Smith and Modrich described the use of the MutSLH complex to remove the majority of errors from PCR fragments (6). In the absence of DAM methylation, the MutSLH complex catalyzes double-stranded cleavage at (GATC) sites. PCR products were treated with MutSLH in the presence of ATP, size-selected to remove digested fragments, and cloned. Treated PCR fragments showed ten-fold reduction in the mutation frequency.

Mismatch-dependent Strand Displacement. Purified E. coli MutS and MutL proteins were reported to activate DNA helicase II in a mismatch-dependent manner (7,8). When a circular DNA molecule with a single nick was treated with MutS, MutL and DNA helicase II Modrich and his coworkers could detect unwinding and the resulting presence of single-stranded DNA only when the DNA molecule contained heteroduplexes. The unwinding proceeded in the direction of the mismatch. This reaction is used herein to preferentially unwind and digest DNA from heteroduplex fragments. The reaction is performed in the presence of Exonuclease I, a single-strand-specific 3′-exonuclease. The synthetic DNA is cloned into a plasmid with a unique nicking endonuclease site adjacent to the cloning site such that the nick is formed on the 3′ side of the cloning site (e.g. N.Bbv C IA, which cuts between the C and the T of the sequence GC*TGAGG). Mismatch-dependent unwinding proceeds towards the mismatched (synthetic) DNA, releasing a single strand with a free 3′end. The single-stranded 3′end is digested by Exonuclease I, resulting in a large single-stranded region on the plasmid and lowering the survival of the molecule during cloning. Alternatively, a brief treatment with mung bean nuclease could be used to digest the single-stranded region and further reduce cloning efficiency of the error-containing molecules.

Removing the nicked DNA. The treatments described above generate molecules containing nicks or small gaps in one strand of the DNA. Exo III is used herein to extend the nicks into larger single-stranded patches. These single-stranded patches facilitate separation of nicked DNA from intact DNA and may reduce the survival of the nicked DNA during cloning. Exo III catalyzes the stepwise removal of mononucleotides from the 3′ termini of duplex DNA including nicked DNA. It is inactive on single-stranded DNA including 3′-protruding termini of four bases or longer. In addition, Exo III is an AP endonuclease and a 3′ phosphatase.

HPLC is used herein to separate nicked DNA from intact DNA, either before or after digestion with Exo III. Partially single-stranded molecules show reduced retention times with ion-pair reverse phase HPLC (this difference is the basis of the DHPLC separations described above). The separation of nicked molecules from intact molecules is used herein under DHPLC conditions. After Exo III digestion, the partially single-stranded molecules are separated from intact molecules under non-denaturing conditions.

In cases where synthetic molecules containing the gaps generated by Exo III digestion of nicks clone far less efficiently than intact molecules, HPLC separation may not be necessary to reduce error rates using this technique.

Genetic Selection Against Error-Containing DNA.

Under certain conditions, the initiation of mismatch repair can lead to cell death. By exploiting these conditions, it is possible to create a strain of E. coli that will not survive when transformed with heteroduplex-carrying plasmids but can be transformed with homoduplex plasmids. The use of a “heteroduplex-killing” strain leads to reduced survival of the error-containing clones and thus a lower error rate in the surviving plasmids.

Based upon a number of recent publications, a genetic selection against heteroduplex-carrying plasmids may be feasible. E. coli strains deficient in all four of the single-strand exonucleases involved in mismatch repair (EXO-) are extremely sensitive to 2-aminopurine, a base analog that is incorporated into DNA and leads to mismatches (9, 10). The sensitivity is dependent on active MutSLH, which suggests that initiating mismatch repair leads to reduced survival. In this model, MutSLH proteins initiate repair at mismatches, but without an active exonuclease the process is diverted into an unproductive pathway, and the cell dies. If MutS is absent, the cells do not initiate mismatch repair, and they survive exposure to 2-aminopurine. If the sensitivity is due to the destruction of the E. coli chromosome after initiation of mismatch repair, this strain may destroy heteroduplex-bearing plasmids and thus reduce the number of errors that survive the cloning process. EXO- and DAM-strains, both of which show MutSLH-dependent sensitivity to 2-aminopurine, are herein used.

Bacteriophage Mu Transposition.

Bacteriophage Mu encodes a mobile genetic element which can insert (or “transpose”) into new sites within a larger DNA molecule. In vitro Mu transposition is reported to exhibit a strong target preference for single-nucleotide mismatches (11). Mu transposition is used herein to alter the size of error-containing synthetic DNA fragments. Heteroduplex molecules are targeted in the present invention by the Mu transposase and receive a Mu insertion. Homoduplex molecules will be less likely to be targets for Mu transposition, and many of these molecules will remain unchanged. If a large fraction of the mismatch-carrying molecules are the target of a transposition reaction, the average molecule that remains the desired size will carry fewer errors than the original population of synthetic DNA molecules.

This approach is limited by the efficiency of in vitro Mu transposition. Even if the specificity is sufficiently high, the sheer number of mutations in synthetic DNA may overwhelm the in vitro transposition system. However, Mu transposition shows a different specificity than many of the other error-detection methods. It does not target one common mutation, small deletions, but does target all eight native mismatches equally. It may be most useful as the final treatment before cloning a gene, after most of the errors have already been removed.

### LITERATURE CITED

1) W. Xiao and P. J. Oefner, Hum. Mutat. 17:439 (2001)

2) Lishanski A, Ostrander E A, Rine J., Proc Natl Acad Sci USA (PNAS) 91:2674-8 (1994)

3) M. Schofield, F. Brownwell, S. Nayak, C. Du, E. Kool, P. Hsieh, Journal of Biological Chemistry 276:45505-45508.

4) X. Pan and S. Weissman, PNAS 99:9346-9351 (2002)

5) A. Ischenko and M. Saparbaev, Nature 415:183-187 (2002)

6) J. Smith and P. Modrich, PNAS 94:6847-6850 (1997)

7) M. Yamaguchi, V. Dao and P. Modrich, Journal of Biological Chemistry 273:9197-9201 (1998)

8) V. Dao and P. Modrich, Journal of Biological Chemistry 273:9202-9207 (1998)

9) V. Burdett, C. Baitinger, M. Viswanathan, S. Lovett, and P. Modrich, PNAS 98:6765-6770 (2002)

10) M. Viswanathan, V. Burdett, C. Baitinger, P. Modrich, and S. Lovett, Journal of Biological Chemistry 276:310523-31058 (2002)

11) K. Yanagihara and K. Mizuuchi, PNAS 99:11317-11321 (2002)

The following examples are offered by way of illustration and not by way of limitation.

### EXAMPLES

### Example 1

### Mismatch Binding with Taq MutS and Gel Analysis

Representatives of the MutS family of proteins are found in a wide variety of organisms, any of which may be useful in this invention. Thermus aquaticus MutS (TaqMutS) is a typical MutS protein, binding loops of 1-4 nucleotides with high affinity as well as all the combinations of mismatched bases with the exception of C to C mismatches. In this example the ability of TaqMutS to bind a defined heteroduplex and removal of the resulting protein-DNA complex is demonstrated.

Mismatch binding experiments were carried out in 10 or 20 ul total volume in 20 mM HEPES pH 7.5, 5 mM MgCl₂, 0.1 mM EDTA, 0.1 mM DTT, 50 ug/ml BSA and 5% (v/v) glycerol. The reaction mixture contained 200 nM of DNA duplex and 1 uM of Taq MutS unless otherwise indicated. The mixture was incubated at 60° C. for 15 minutes and cooled to 4° C. Gel shift analysis was done on 5% acrylamide gel cast in 1× TBE and 10 mM MgCl₂.

Gel shift assays with Taq MutS protein and a set of synthetic 50 bp homoduplex and heteroduplex fragments were consistent with the literature. There was observed nearly quantitative binding to one- or two-bp insertions/deletions (lanes 3 and 15) and a much less complete shifting of the mismatch substrates. A- and T-containing bulges were bound well (lanes 3, 20 and 22), but G- and C-containing bulges were shifted much less effectively (lanes 21 and 23).

### Example 2

### Binding of TaqMutS to Defined Test Heteroduplex DNA and Removal of Protein-DNA Complexes

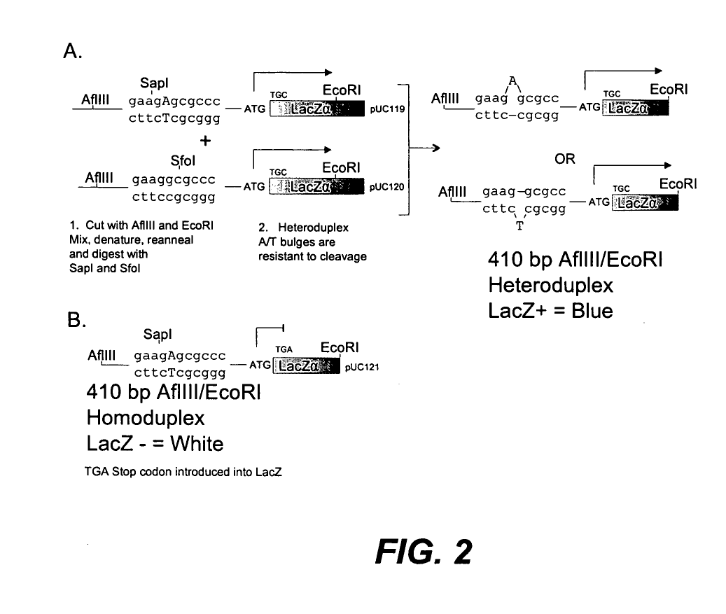

A test heteroduplex fragment linked to a gene fragment that results in a blue colony phenotype when cloned directionally into a pUC vector was generated. A 410 bp AflIII/EcoRI fragment that included the start codon and 5′ coding region for an active LacZα gene was generated containing a single A or T deletion heteroduplex upstream of the LacZα gene. The same homoduplex 410 bp fragment was created with a single base change resulting in a stop codon in the 5′ coding region of the LacZα gene. In this way the heteroduplex fragments are linked to an active fragment of the LacZα gene, while the homoduplex molecules are linked to an inactive LacZα gene fragment. Ligation of the active or inactive N-terminal LacZα fragment to restore a complete LacZα gene allows heteroduplex or homoduplex molecules to be scored by counting blue or white colonies when grown on media containing X-Gal. The scheme for generating the heteroduplex substrate is shown in FIG. 2. Mixing homoduplex and heteroduplex fragments in a defined ratio allows the blue (heteroduplex) and white (homoduplex) colonies to be scored following ligation into a pUC vector, electroporation and plating of transformants on LB+Amp+Xgal agar plates.

The 410 bp white:blue test heteroduplex was used to determine the best conditions for separation of a model A or T deletion heteroduplex from perfectly matched homoduplex molecules using TaqMutS. A defined ratio of heteroduplex and homoduplex 410 bp fragments were incubated with TaqMutS at 60° C. for 20 minutes and subsequently passed through enzyme removal columns (Micropure-EZ enzyme removers from Millipore). These columns are marketed as quick alternatives to phenol/chloroform methods for removing proteins from DNA. The aim was to retain the TaqMutS protein bound to heteroduplex DNA in the column and retrieve the homoduplex DNA in the flow-through. It was observed that at the predetermined optimal concentrations of 500 nM TaqMutS and 40 nM white:blue test DNA, the fraction obtained that flowed through the column resulted in a shift of white:blue colony ratio from 1:1 to 60:1 when cloned and plated on LB agar plates containing a drug to select the transformants and the X-Gal substrate. Greater than 98% of the A or T bulged heteroduplex molecules (blue colonies) were removed by the Micropure-EZ enzyme removal column under these conditions.

### Example 3

### Binding of TaqMutS to a 354 BP Synthetic DNA and Removal of Protein-DNA Complexes

Direct binding of TaqMutS to synthetic DNA was determined as follows. 500 nM TaqMutS was incubated with 40 nM 354 bp synthetic DNA at 60° C. for 20 minutes. DNA obtained following treatment with Micropure-EZ enzyme removal columns (Deproteination), was cloned and sequenced. The results are displayed below in Table 2.

Only 2 out of 15 clones sequenced in the no treatment control group had the correct sequence, representing an error frequency of 1/212 base pairs. The Micropure-EZ enzyme removal column flow-through deproteinated fraction showed substantial improvements (1/1593 P<0.001). Over 85% of all errors were removed in the Micropure-EZ column deproteinated fraction when compared to the no treatment control DNA.

TABLE 2
TaqMutS binding and removal of synthetic DNA-protein complexes

| | | | | | Ave. # | Error | |
|---|---|---|---|---|---|---|---|
| | # Fully | # Correct | # Total | % | Error/ | Frequency | |
| Treatment | Sequenced | Sequence | Errors | Correct | fragment | (1/x bp) | P value |
| Deproteination | 18 | 15 | 4 | 83.3 | 0.2 | 1593 | <0.0001 |
| No treatment control | 15 | 2 | 25 | 13.3 | 1.7 | 212 | |

### Example 4

### Introduction of a Nick into Heteroduplex Test DNA, Labeling with Biotin and Removal of Protein-Biotin-DNA Complexes

The 410 bp white:blue test heteroduplex (FIG. 2, Example 2 above) was used to determine the best conditions for separation of a model A or T deletion heteroduplex from perfectly matched homoduplex molecules using the CELI endonuclease. CELI endonuclease is known by those skilled in the art to recognize heteroduplexes of a variety of kinds, including flaps, cruciform junctures, bulged DNA and mismatched bases. A single strand 3′-OH nick is formed at or near the site of the alternate DNA structure. The 3′OH nick is substrate for DNA polymerase which can incorporate biotinylated dUTP into the nicked DNA molecules. Overhanging ends are substrate for mismatch endonucleases, so linear fragments cannot be used. Close circular plasmids were generated by ligation of the heteroduplex (white) or homoduplex (blue) molecules into pUC119 digested with AflIII and EcoRI restriction endonucleases. Ligated DNAs were mixed at a 1:1 ratio before treatment with 0.2 Units of the CELI mismatch endonuclease for 30 minutes at 30° C. in a final volume of 20 ul. Following treatment BstL DNA polymerase and dNTP's were added including Biotin-dUTP and the reaction was heat treated at 65° C. for 20 minutes to destroy the CELI activity and incorporate biotin into the nicked molecules. Biotin-dUTP is known by those skilled in the art to be incorporated into nicked DNA by BstL DNA polymerase. A 5 fold molar excess of streptavidin to biotin was added and the reaction was incubated for 20 minutes at room temperature. Plasmid DNA obtained following treatment with Micropure-EZ enzyme removal column was transformed into E. coli and plated onto LB agar+ampicillin+X-Gal. Control reactons were performed without adding CELI or biotin-dUTP or without addition of polymerase. The control reactions yielded blue and white colonies at the expected ratio of 1:1 while the CELI treated reaction with polymerase and biotin-dUTP resulted in a shift in ratio from 1:1 to 1:5 blue to white colonies. This indicates that greater than 80% of the A or T bulged heteroduplex DNA became associated with a protein-biotin-DNA complex and was removed following deproteinization of the solution.

### Example5

### Treatment of 354 BP Synthetic DNA with CELI Endonuclease, Incorporation of Biotin and Removal of Protein-Biotin-DNA Complexes

Synthetic DNA was cloned into pUC119 and treated with 0.2 Units CELI endonuclease for 30 minutes at 30° C. in a 20 ul reaction volume. Following treatment BstL DNA polymerase and dNTP's were added including Biotin-dUTP and the reaction was heat treated at 65° C. for 20 minutes to destroy the CELI activity and to incorporate biotin into the nicked molecules. A 5 fold molar excess of streptavidin to biotin was added and the reaction was incubated for 20 minutes at room temperature. Protein-biotin-DNA complexes were removed by treatment with Micropure-EZ enzyme removal columns (Deproteination). The deproteinized flow through fraction was transformed into E. coli and plated onto LB agar+ampicillin. Colonies representing single clones were sequenced and the error frequencies determined. The results are displayed below in Table 3. CELI treatment reduced the error frequency from 1 error in 212 base pairs to 1 error in 472 base pairs (P=0.0137 Chi squared).

TABLE 3
CELI endonuclease treatment and removal of protein-biotin-DNA
complexes from synthetic DNA

| | | | | | Ave. # | Error | |
|---|---|---|---|---|---|---|---|
| | # Fully | # Correct | # Total | % | Error/ | Frequency | |
| Treatment | Sequenced | Sequence | Errors | Correct | fragment | (1/x bp) | P value |
| CEL1 | 12 | 6 | 9 | 50.0 | 0.8 | 472 | 0.0137 |
| No treatment | 15 | 2 | 25 | 13.3 | 1.7 | 212 | |

### Example 6

### MuA Transposase Strand Transfer of Biotin Labeled MuA End DNA into Synthetic DNA

MuA catalyzed DNA cleavage and joining reactions resulting in strand transfer can be promoted in vitro using as little as 51 bp of precleaved MuA right end DNA. This reaction has been shown to occur specifically at mismatched DNA sites for all mismatch combinations, and to a lesser extent at G bulges. This targeted transposition reaction was used to insert a biotinylated MuA right end DNA fragment into mismatched synthetic DNA, bind biotin with streptavidin and separate the DNA/protein complexes using the Micropure-EZ enzyme removers from Millipore. The DNA obtained was ligated into pUC119 and transformed into E. coli. Clones were picked and sequenced.

No base substitutions were observed in a total of 8496 bp sequenced for the MuA treated synthetic DNA. This contrasts with the frequency of 1 base substitution per 1770 bp for the untreated control (P=0.0284 Chi squared analysis). The frequency of deletions was not significantly improved from 1/241 to 1/315, as expected. MuA transposition has limited specificity for G bulged DNA and shows no preference for insertion into other single or multiple bulged DNA sites.

From the foregoing it will be appreciated that, although specific embodiments of the invention have been described herein for purposes of illustration, various modifications may be made without deviating from the spirit and scope of the invention.

1. A method of depleting in a sample of double-stranded oligonucleotides a population of double-stranded oligonucleotides containing mismatched bases thereby enriching in said sample a population of double-stranded oligonucleotides containing correctly matched bases, comprising the steps of:
(a) contacting said sample containing double-stranded oligonucleotides with a mismatch recognition protein under conditions to permit the protein to interact with a double-stranded oligonucleotide containing at least one mismatched base; and
(b) collecting double-stranded oligonucleotides that have not interacted with said mismatch recognition protein, thereby depleting the population of double-stranded oligonucleotides containing mismatched bases.

2. The method of claim 1 wherein prior to the step of collecting, having an additional step comprising separating said double-stranded oligonucleotide containing at least one mismatched base that has interacted with said mismatch recognition protein, from double-stranded oligonucleotides that have not interacted with said mismatch recognition protein.

3. The method of claim 1 wherein the double-stranded oligonucleotides of said sample are chemically synthesized.

4. The method of claim 1 wherein the double-stranded oligonucleotides of said sample are enzymatically synthesized.

5. The method of claim 4 wherein prior to the step of contacting, having additional steps comprising denaturing and reannealing said sample of double-stranded oligonucleotides under conditions to permit conversion of the double-stranded oligonucleotides first to single-stranded oligonucleotides and then to double-stranded oligonucleotides.

6. The method of claim 1 wherein said mismatch recognition protein is immobilized on a solid support.

7. The method of any one of claims 1, 2, 3, 4, 5 or 6 wherein said double-stranded oligonucleotides are double-stranded DNA.

8. The method of claim 7 wherein the DNA is a gene or a portion of a gene.

9. The method of claim 1 or claim 2 wherein said mismatch recognition protein is MutS.

10. The method of claim 1 or claim 2, wherein immediately following step (a) or simultaneous with step (a), contacting said sample with a nucleotide containing biotin under conditions to permit incorporation of the nucleotide into the oligonucleotides that have interacted with said mismatch recognition protein.

11. The method of claim 10 wherein said mismatch recognition protein is CELI endonuclease.

12. The method of claim 10 wherein said mismatch recognition protein is MuA transposase.

13. The method of claim 10 wherein, following contacting said sample with a nucleotide containing biotin, said sample is contacted with an avidin under conditions to permit the avidin to interact with the biotin.

14. The method of claim 13 wherein said avidin is immobilized on a solid support.

15. The method of claim 13 wherein said avidin is streptavidin.

16. A kit for depleting double-stranded oligonucleotides containing mismatched bases from a population of double-stranded oligonucleotides, comprising a mismatch recognition protein, buffer, control oligonucleotides and instructions.

17. The kit of claim 16, further comprising material for separating mismatch protein bound oligonucleotides from unbound oligonucleotides.

18. The kit of claim 16 or claim 17 wherein said double-stranded oligonucleotides are double-stranded DNA.

19. The kit of claim 18 wherein the DNA is a gene or a portion of a gene.


SEQUENCE LISTING (6 sequences)

SEQ ID NO: 1 - DNA, 11 bp, Artificial Sequence
  Fragment of pUC119
gaagagcgccc

SEQ ID NO: 2 - DNA, 11 bp, Artificial Sequence
  Fragment of pUC119
cttctcgcggg

SEQ ID NO: 3 - DNA, 10 bp, Artificial Sequence
  Heteroduplex DNA containing single A bulge
gaagagcgcc

SEQ ID NO: 4 - DNA, 10 bp, Artificial Sequence
  Fragment of pUC120
gaaggcgccc

SEQ ID NO: 5 - DNA, 11 bp, Artificial Sequence
  Fragment of pUC120
cttctcgcggg

SEQ ID NO: 6 - DNA, 10 bp, Artificial Sequence
  Heteroduplex DNA containing single T bulge
cttctcgcgg